(19) United States
(12) Patent Application Publication
(10) Pub. No.: US 2019/0076676 A1
(43) Pub. Date: Mar. 14, 2019

(54) TISSUE BONDING COPOLYMERS OF OIL

(71) Applicant: MAPLE RIDGE, LLC, New York, NY (US)

(72) Inventors: Michael Milbocker, Holliston, MA (US); Kenneth O. Rothaus, New York, NY (US)

(21) Appl. No.: 16/125,692

(22) Filed: Sep. 8, 2018

Related U.S. Application Data
(60) Provisional application No. 62/556,106, filed on Sep. 8, 2017.

(51) Int. Cl. A61Q 5/12; A61K 8/37; A61K 8/92

(52) U.S. Cl. CPC A61Q 5/12; A61K 8/92; A61K 8/375

(57) ABSTRACT

The present disclosure provides a keratin care composition, such as a hair rinse, hair masque, or hair serum composition comprising, a supramolecular or molecular compound that may be obtained by reaction between: i) at least two oil molecules each containing at least one reactive group chosen from OH, —CO(O)H, and NH2, ii) at least one linking molecule comprising at least two isocyanate or imidazole groups, and iii) said linking molecule comprising at least one pendant group capable of bonding to protein. The linking molecule may comprise a block ethylenic copolymer. Preferably, the block ethylenic copolymer is comprised of ethylene and propylene blocks. The bonded protein is generally keratin, and in particular, hair and/or skin.

### CROSS-REFERENCE TO RELATED APPLICATIONS

This application claims benefit of priority to U.S. provisional application No. 62/556,106, filed on Sep. 9, 2017, the contents of which are hereby incorporated by reference in their entirety.

### TECHNICAL FIELD

The present disclosure generally relates to a cosmetic composition, in particular hair or skin treatment containing a copolymer exceptionally effective in enhancing the keratin conditioning effect.

### BACKGROUND OF THE INVENTION

Keratin is one of a family of fibrous structural proteins. Keratin is the protein that protects epithelial cells from damage or stress. It is the key structural material making hair. The compositions of the present invention are capable of bonding to keratin.

In recent years, damage control products such as high-grade rinse, masque and out-bath serum treatment illustrate that consumer awareness of damage to hair is increasing. For reducing the damaged feel (enhancing the touch feel) of hair, a masque (conditioner) is used after washing hair with a rinse (shampoo), and when the damaged feel still remains, a treatment known as a serum is applied and rinsed out shortly thereafter or may be left on the hair for a longer period of time.

The damaged feel of hair is expressed mainly by sensory evaluation of, for example, a friction between hairs or between hair and hand or a parched look or hardness due to lack of moisture. One approach to improve the damaged feel of hair is to apply a treatment containing a cationic surfactant, such as stearyl trimethylammonium chloride, for reducing the friction and a softener component for softening the hair. Nevertheless, such treatments do not bond to the hair, and quickly wash out.

There remains a need for compositions for repairing hair, particularly damaged hair. In particular, damaged hair cuticle needs repair. The cuticle is comprised of the protein keratin in a superficial layer of overlapping cells covering the hair shaft (cuticula pili) that locks the hair into its follicle. When the cuticle layer is damaged, the keratin structures can open resulting in friction between hairs and the damaged feel.

Synthetic thickening polymers in the form of inverse lattices are known in the art. However, most of these thickeners are incapable of thickening oil phases. Cosmetic compositions in the form of water-in-oil emulsions containing as thickener a polymer with a low proportion of units containing ionic groups, for instance the copolymer of N-dodecylacrylamide and of 2-acrylamido-2-methylpropanesulfonic acid in a 96.5/3.5 weight proportion or the copolymer of N-tert-butylacrylamide and of 2-acrylamido-2-methylpropanesulfonic acid in a 97.9/2.1 weight proportion. Such copolymers are difficult to use in the cosmetic industry since they must be neutralized before use, and since their dissolution in oils often requires the use of a cosolvent. Furthermore, their oil-thickening capacity is low.

It is for this reason that the only compounds known to date having oil-thickening capacity property are the copolymers sold under the name Intelimer, which are hydrophobic copolymers bearing long pendent alkyl chains, which crystallize under cold conditions to form clusters that bring about thickening of the medium. Such polymers of the type such as copolymers of an alkyl acrylate and of (meth)acrylic acid are described in the American patents published under the numbers U.S. Pat. No. 7,101,928 B1 and U.S. Pat. No. 5,736,125.

These products are not easy to use since the polymer needs to be dissolved in the oil under hot conditions, followed by cooling to bring about crystallization of the polymeric chains. Furthermore, they are by nature heat-sensitive and compositions thickened with a polymer of this type are difficult to market in hot countries.

Most rinse or masque treatments require washing after application to hair, but according to numerous studies, most of the softener component flows out during rinsing. Consequently, a higher conditioning effect is recently demanded. Furthermore, when a rinse and masque treatment can produce a high treatment effect without requiring repeated use, the burden in terms of cost on the consumer can be lessened. For this reason, demands for such improvement on a hair cosmetic are increasing.

Accordingly, there is a need for keratin treatment compositions that are easy to use and apply and that remain in or attached to the hair, without being easily rinsed out, thus providing a longer lasting effect.

### BRIEF SUMMARY OF THE INVENTION

An object of the present disclosure is to solve those problems and provide a copolymer capable of realizing a hair cosmetic ensuring that a softener component in the form of a di-oil can persist on hair even after water rinsing. The present invention achieves enhance softness without compromising the feel of smoothness. An excellent conditioning effect can be produced without impairing the feel such as smoothness, using a cosmetic composition and a hair cosmetic containing a copolymer of oil.

A di-oil according to the present disclosure is a covalently bonded or hydrogen bonded composition comprising a linking molecule and two or more oil molecules. Preferably, the linking molecules contains a free pendant group capable of bonding to keratin. When the oils are hydrogen bonded to the linking molecule the resulting compound is known as a supramolecular compound.

The di-oil may be combined with additional ingredients preferably chosen from: silicone elastomers, film-forming polymers, silicone resins, polycondensates, semicrystalline polymers, thickeners comprising at least one group and preferably at least two groups capable of establishing hydrogen interactions, chosen from polymeric thickeners and organic gelling agents.

Many cosmetic compositions exist for which gloss properties of the deposited film, after application to keratin materials, are desired. For example, in the present invention, the protein bonding functionality bonds di-oil directly to the cuticle, sealing the cuticle thereby reducing friction, and adding sheen.

Many oils, such as argan, jojoba, flax and olive possess particularly effective hair repair characteristics. When the oil absorbs into the cuticle the hair shaft increases in volume and the cuticle layer closes. Paradoxically, such oils reduce the total volume of the hair by causing individual hairs to stick together. It is particularly difficult to uniformly treat the hair without obtaining a greasy look. These oils are commonly characterized as being thick, and matte in appearance.

Other oils, such as prickly pear, grape seed, rose and pomegranate possess particularly effective gloss characteristics. Nevertheless, mechanical mixtures of gloss and repair oils possess poor persistence in the hair, and separate when heated or stored.

It was unexpectedly discovered by the inventors that many di-oils constructed according to the teaching of the present application act synergistically. Di-oils of the present invention possess superior reparative, feel, and look characteristics when compared to mechanical mixtures of the same oils. Accordingly, many of the advantages realized in the present application do not relying on the protein bonding functionality.

In addition, compositions containing look and feel additives, which do not possess pendant OH or NH2 groups, and hence cannot form di-oils of the present invention, can beneficially be associated to or locked in the hair by the protein bonding functionality of the di-oils. Examples of additional composition additives include especially lanolins, and “glossy” oils such as polybutenes, or esters of fatty acid or of fatty alcohol with a high carbon number; or alternatively certain plant oils; or alternatively esters resulting from the partial or total esterification of a hydroxylated aliphatic compound with an aromatic acid.

Alternatively, hair care compositions may be provided in two separate parts to be mixed upon administration to hair. One part can be a di-oil stable composition, and the other part a composition that may interact with the di-oil composition. In particular, one part may be applied while the hair is liberally wet and the other part applied with the hair is towel dry. In particular, the first part may chemically bond with the second part. The later feature may be particularly useful in obtaining a hair treatment that is not overly treated in either of the components. For example, when the first part bonds to hair but retains some bonding functionality in the form of unreacted isocyanate, imidazole or water transformed NH2 groups, these unreacted groups react to a second part in a proportion that reflects the hair care need, or a desired look or feel outcome.

When the di-oil is held together by supramolecular chemistry, the chemistry involves groups of molecules loosely bound in a desired configuration. The forces responsible for the spatial organization may vary from weak intermolecular forces, electrostatic or hydrogen bonding to strong covalent bonding, provided that the degree of electronic coupling between the molecular component remains small with respect to relevant energy parameters of the component.

Supramolecular chemistry is to be contrasted with traditional chemistry which focuses on the covalent bond. Supramolecular chemistry examines the weaker and reversible noncovalent interactions between molecules. These forces include hydrogen bonding, metal coordination, hydrophobic forces, van der Waals forces, pi-pi interactions and electrostatic effects.

In some embodiments, the present disclosure provides a keratin care composition comprising, in a keratin care acceptable medium, a compound obtained by the reaction of: i) at least two oil molecules each containing at least one functional group selected from the group consisting of —OH, —C(O)OH, —NH₂, and combinations thereof and ii) at least one linking molecule comprising at least two isocyanate or imidazole groups capable of reacting with the reactive functional groups on the oil molecules, wherein the linking molecule further comprises at least one pendant group capable of bonding to a protein.

In other embodiments, the present disclosure provides A keratin care composition comprising, in a keratin care acceptable medium, a compound obtained by the reaction of: i) at least two oil molecules each containing at least one functional group selected from the group consisting of —OH, —C(O)OH, —NH₂, and combinations thereof and ii) at least one linking molecule comprising at least two isocyanate or imidazole groups capable of reacting with the reactive functional groups on the oil molecules. In this embodiment, the composition does not contain a protein bonding group. In certain embodiments, the composition comprises a mixture of protein bonding and non-protein bonding compounds disclosed herein.

In some embodiments, the linking group is a multi-armed polymer, such as a multi-armed polaxamer. In certain embodiments, the linking molecule comprises isocyanate groups that react with the functional group on the oil. For example, the polymer may be a three-armed polymer with three isocyanate groups comprises three isocyanate groups.

In some embodiments, the functional groups on the at least two oils are —OH, CO₂H or a combination thereof.

The at least two oils are the same or different and are selected from the group consisting of: a saturated or unsaturated, linear or branched C₆-C₅₀ monoalcohol; a saturated or unsaturated, linear or branched C₆-C₅₀ diol; a saturated or unsaturated, linear or branched C₆-C50 triol; linear or branched C₆-C₅₀ fatty acid; a pentaerythritol partial ester; a dipentaerythritol diester, triester, tetraester, or pentaester.

The linking molecule comprises a backbone molecule selected from the group consisting of: trimethylolpropane monoester, trimethylolpropane monoester diester; a bis(trimethylolpropane) monoester, a bis(trimethylolpropane) monoester diester, a bis(trimethylolpropane) monoester triester; an ester of glycerol, an ester of polyglycerol; a propylene glycol monoester; a diol dimer monoester; a glycerol ether; an ester of a hydroxylated monocarboxylic acid, an ester of a hydroxylated monocarboxylic an ester of a dicarboxylic acid, an ester of a hydroxylated tricarboxylic acid; a triglyceryl ester comprising an OH; a hydrogenated castor oil, a non-hydrogenated castor oil, and a poloxamer.

In certain embodiments, the linking molecule comprises three terminal groups, two terminal groups of which are bonded to the oil molecules, and the third terminal group is a free isocyanate group, which is capable of reacting with a protein, such as keratin. The at least one protein bonding terminal group may be an isocyanate group derived by the reaction of an —OH or —NH₂ group on the linker molecule with toluene diisocyanate, isophorone diisocyanate, methylenebis(phenyl isocyanate), hexamethylene diisocyanate, naphthalene diisocyanate, and methylene bis-cyclohexylisocyanate.

In certain embodiments, the oil has a refractive index of greater than or equal to 1.46 at 25° C. In some embodiments, the oil has a molecular weight (Mw) ranging from 150 to 6000 g/mol. The oil may be selected from the group consisting of: a linear, branched, or cyclic, saturated or unsaturated fatty alcohol comprising 6 to 50 carbon atoms and an OH group, and optionally an NH₂; and an ester of a hydroxylated dicarboxylic acid with a monoalcohol.

In particular embodiments, the oils are the same or different and are selected from the group consisting of argan oil, oleic acid, citronellol, trichosanic acid, linoleic acid and menthol.

In some embodiments, the linker molecule contains the radical —R¹—O—C(O)—NH—R²—NCO, wherein R¹ and R² are each independently a divalent carbon-based radical selected from the group consisting of a linear or branched alkyl group, a cycloalkyl group, and an aryl group; or a mixture thereof. More particularly, the linker is obtained by the reaction of a polyethylene/polypropylene copolymer (such as a polaxamer) with at least three diisocyanate molecules.copolymer is a poloxamer.

In some embodiments, the number-average molecular mass (Mn) of the compound is from 180 to 8000.

In certain embodiments, the compound is present in the composition in an amount ranging from 5% to 95% by weight, relative to a total weight of the composition.

The composition may further comprise a semicrystalline polymer selected from the group consisting of: a homopolymer or copolymer comprising a unit obtained from polymerizing a monomer comprising a crystallizable hydrophobic side chain; a polymer comprising, in the backbone, a crystallizable block; a polycondensate of an aliphatic, an aromatic, or an aliphatic/aromatic polyester; at least one selected from the group consisting of an ethylene homopolymer, a propylene homopolymer, and an ethylene/propylene copolymer prepared via metallocene catalysis.

In other embodiments, the composition further comprises a thickener selected from the group consisting of: (i) a polymer having a weight-average molecular mass of less than 100,000, and comprising a) a polymer backbone comprising hydrocarbon repeating units comprising a heteroatom, and optionally comprising b) at least one selected from the group consisting of a pendent fatty chain and a terminal fatty chain, optionally functionalized, comprising from 6 to 120 carbon atoms, and linked to the hydrocarbon; and (ii) a silicone polyamide.

In still other embodiments, the composition further comprising: at least one additive selected from the group consisting of a volatile oil, a nonvolatile oil, a dyestuff, a pasty fatty substance, a wax, and a filler.

The present disclosure further comprises a method for treating a keratin material, the process comprising: applying the cosmetic composition of claim 1 to a keratin material.

One subject of the present invention is thus a cosmetic composition for caring for keratin materials (especially the skin or the hair), comprising, in a cosmetically acceptable medium:

(i) a di-oil compound that can be obtained by reaction between: at least two oil molecules possessing each at least one nucleophilic reactive function chosen from OH and NH2, and at least one linker group capable of establishing bonds with at least two oil molecules, each pairing of a linker group involving at least two bonds with oil molecules, the said linker group bearing at least one isocyanate or imidazole reactive function capable of reacting with the reactive function on the oil molecules, the said linker group also comprising at least one pendant group of structure:

—R¹—O—C(O)—NH—R²—NCO

in which: R¹ and R², which may be identical or different, represent a divalent carbon-based radical chosen from (i) a linear or branched alkyl group, (ii) cycloalkyl group, (iii) an aryl group; optionally substituted with an ester or amide function or with an alkyl radical; or a mixture of these groups and (iv) a multi-armed poloxamer.

(ii) optionally at least one additional non aqueous ingredient preferably chosen from: silicone elastomers, linear or branched monocarboxylic acid; a cyclic dicarboxylic acid; and an aromatic monocarboxylic acid, film-forming agents chosen from silicone resins and film-forming polymers, preferably chosen from the group comprising: a film-forming block ethylenic copolymer, a vinyl polymer comprising at least one carbosilane dendrimer-based unit, a dispersion of acrylic or vinyl radical homopolymer or copolymer particles dispersed in the said liquid fatty phase, structuring agents chosen from semicrystalline polymers and thickeners comprising at least one group and preferably at least two groups capable of establishing hydrogen interactions, chosen from: polymeric thickeners, and organic gelling agents.

The di-oils when functionalized with a protein bonding pendant group according to the present invention are in the form of a liquid; which convert to a solid when bonded to protein, this makes it possible especially to form a non-tacky material, which does not transfer onto the fingers once applied to keratin materials.

Moreover, it has been found that increasing the number of pendant protein bonding arms on the di-oil can increase the strength of crosslinking to protein and other di-oils. For example, the pendant isocyanate group can convert to an amine group when exposed to water, the formed amine group can react with another pendant isocyanate group on another di-oil, thereby chain extending the di-oil bonded to keratin. Increasing the number of isocyanate pendant arms improves the permanence of the desired cosmetic effect, most particularly the permanence of the deposit or of the gloss or reparative oils.

Furthermore, the di-oils are easy to convey in the usual cosmetic media, especially the usual cosmetic oily or nonaqueous media, for example especially oils, fatty alcohols and/or fatty esters, which facilitates their use in the cosmetic field, especially in serums. The di-oils show acceptable solubility in varied cosmetic oily media, such as plant oils, alkanes, esters, whether they are short esters such as butyl or ethyl acetate, or fatty esters, and fatty alcohols, and most particularly in media comprising isododecane, parleam, isononyl isononanoate, octyldodecanol and/or a C12-C15 alkyl benzoate.

They are advantageously compatible with the oils usually present in cosmetic compositions, and also have good properties of dispersing pigments or fillers.

The di-oils of the present invention are also useful in solid compositions. The cosmetic compositions according to the invention moreover show good applicability (glidance on application and decaking in the case of solid compositions) and good coverage; good adherence to the support, whether it is to the nails, the eyelashes, the skin or the lips; adequate flexibility and strength of the film, and also an excellent gloss durability. The comfort and glidance properties are also very satisfactory.

In conclusion, the di-oils of the compositions according to the invention thus comprise at least one part (O) originating from the oil and at least one part (L) originating from the linker molecule, and optionally a part (B) attached to the linker molecule capable of bonding to protein, the said part (B) comprising at least one unit of formula

—R¹—O—C(O)—NH—R²—NCO

where R¹ and R² are as defined above.

In particular, the said parts (O) and (L) are connected via a covalent bond and may especially be connected via a covalent bond formed during the reaction between the OH and/or NH2 reactive functions on the oil with the isocyanate reactive functions borne by the linker molecule; or alternatively between the NH2 reactive functions borne by the oil and the isocyanate or imidazole functions borne by the linker molecule

### DETAILED DESCRIPTION OF THE INVENTION

Oils useful in the present invention include any botanically derived oil. The examples of which are given below.

Oil of argan, prickly pear seed, and flax seed:A principle component of argan, prickly pear, and flax seed oil is oleic acid. Oleic acid is a fatty acid that occurs naturally in various animal and vegetable fats and oils. It is an odorless, colorless oil, although commercial samples may be yellowish. In chemical terms, oleic acid is classified as a monounsaturated omega-9 fatty acid, abbreviated with a lipid number of 18:1 cis-9. It has the formula CH₃(CH₂)₇CH═CH(CH₂)₇COOH. The term “oleic” means related to, or derived from, olive oil which is predominantly composed of oleic acid.

The stereoisomer of oleic acid is called elaidic acid or trans-9-octadecenoic acid which are also useful in the present invention. These isomers have distinct physical properties and biochemical properties. Elaidic acid, the most abundant trans fatty acid in diet, appears to have medicinal properties.

Another naturally occurring isomer of oleic acid is petroselinic acid.

In chemical analysis, fatty acids are separated by gas chromatography of their methyl ester derivatives. Alternatively, separation of unsaturated isomers is possible by argentation thin-layer chromatography.

Another oil components is linoleic acid, a polyunsaturated omega-6 fatty acid. It is a colorless liquid at room temperature. In physiological literature, it has a lipid number of 18:2 cis,cis-9,12. Linoleic acid is a carboxylic acid with an 18-carbon chain and two cis double bonds; with the first double bond located at the sixth carbon from the methyl end.

Linoleic acid (LA) belongs to one of the two families of essential fatty acids, which means that the human body cannot synthesize it from other food components. LA is a polyunsaturated fatty acid used in the biosynthesis of arachidonic acid. It is found in the lipids of cell membranes. It is abundant in many nuts, fatty seeds (flax seeds, hemp seeds, poppy seeds, sesame seeds, etc.) and their derived vegetable oils; comprising over half (by weight) of poppy seed, safflower, sunflower, corn, and soybean oils.

LA is converted by various lipoxygenases, cyclooxygenases, certain cytochrome P450 enzymes (the CYP monooxygenases), and non-enzymatic autoxidation mechanisms to mono-hydroxyl products viz., 13-Hydroxyoctadecadienoic acid and 9-Hydroxyoctadecadienoic acid; these two hydroxy metabolites are enzymatically oxidized to their keto metabolites, 13-oxo-octadecadienoic acid and 9-oxo-octadecdienoic acid. These conversion process can occur on the copolymers of the present invention.

Other conversion enzymes include cytochrome P450 enzymes, the CYP epoxygenases, metabolize LA to epoxide products viz., its 12,13-epoxide, Vernolic acid and its 9,10-epoxide, Coronaric acid.

All of these LA products have bioactivity and are active in human physiology health and pathology prevention. Linoleic acid is an essential fatty acid that must be consumed for proper health. A diet only deficient in linoleate (the salt form of the acid) causes mild skin scaling, hair loss, and poor wound healing in rats.

Other sources of linoleic acid useful in the present invention include: salicornia oil, safflower oil, evening primrose oil, poppyseed oil, grape seed oil, sunflower oil, barbary fig seed oil, hemp oil, corn oil, wheat germ oil, cottonseed oil, soybean oil, walnut seed oil, rice bran oil, pistachio oil, peanut oil, peach oil, almond oil, canola oil, olive oil, palm oil, cocoa butter oil, macadamia nut oil, coconut oil.

Palmitic acid is also useful in the present invention. Hexadecanoic acid in IUPAC nomenclature, is the most common saturated fatty acid found in animals, plants and microorganisms. Its chemical formula is CH3(CH2)14COOH. As its name indicates, it is a major component of the oil from the fruit of oil palms (palm oil).

Palmitic acid can also be found in meats, cheeses, butter, and dairy products. Palmitate is a term for the salts and esters of palmitic acid, and can be useful as delivery agents of the present invention. The palmitate anion is the observed form of palmitic acid at physiologic pH (7.4).

Stearic acid, also useful in the present invention, is a saturated fatty acid with an 18-carbon chain and has the IUPAC name octadecanoic acid. It is a waxy solid and its chemical formula is C₁₇H₃₅CO₂H.

The salts and esters of stearic acid are called stearates, and are also useful in the present invention. As its ester, stearic acid is one of the most common saturated fatty acids found in nature following palmitic acid. The triglyceride derived from three molecules of stearic acid is called stearin. Stearin is particularly useful as a diluent in the present invention comprising copolymerized oils.

In general, the copolymeric applications of stearic acid are appreciated in that such constructs display a bifunctional character. The polar head group of stearic acid can be attached to metal cations and a nonpolar chain that confers solubility in organic solvents. This functionality can make oil copolymers of the present invention soluble in aqueous preparations. A feature important in cosmetic and healthcare products. For example, the stearic acid cation combination leads to uses as a surfactant and softening agent.

Stearic acid undergoes the typical reactions of saturated carboxylic acids, a notable one being reduction to stearyl alcohol, and esterification with a range of alcohols. Stearic acid and these reactions are useful in thickening cosmetic compositions.

Stearic acid is mainly used in the production of detergents, soaps, and cosmetics such as shampoos and shaving cream products. Copolymerizing stearic acid with other oils enhances these products. Soaps are not made directly from stearic acid, but indirectly by saponification of triglycerides consisting of stearic acid esters. Copolymerization of these saponification products are useful in the present invention.

Esters of stearic acid with ethylene glycol, glycol stearate, and glycol distearate are used to produce a pearly effect in shampoos, soaps, and other cosmetic products. That pearl effect can be enhanced by copolymerization with other oils.

Copolymerized stearic acid can be added to product in molten form and allowed to crystallize under controlled conditions. Detergents obtained from amides and quaternary alkylammonium derivatives of stearic acid can be copolymerized in the present invention.

Rose oil: The following are oils found in rose that can polymerized according to the present invention to produce copolymers with characteristics useful in hair care products. Citronellol, or dihydrogeraniol, is a natural acyclic monoterpenoid. Citronellol occurs in two enantiomeric forms. Both enantiomers occur in nature. (+)-Citronellol, which is found in citronella oils, including Cymbopogon nardus, is the more common isomer. (−)-Citronellol is found in the oils of rose and Pelargonium geraniums. Both are useful in the present invention.

Geraniol is a monoterpenoid and an alcohol. It is the primary part of rose oil, palmarosa oil, and citronella oil. It also occurs in small quantities in geranium, lemon, and many other essential oils. It appears as a clear to pale-yellow oil that is insoluble in water, but soluble in most common organic solvents. It has a rose-like scent and is commonly used in perfumes. It is used in flavors such as peach, raspberry, grapefruit, red apple, plum, lime, orange, lemon, watermelon, pineapple, and blueberry.

Nerol is a monoterpene found in many essential oils such as lemongrass and hops. It was originally isolated from neroli oil. Nerol is a colourless liquid used in perfumery. Nerol is isomeric with geraniol, the double bond is trans.

Linalool refers to two enantiomers of a naturally occurring terpene alcohol found in many flowers and spice plants. Linalool has other names such as β-linalool, linalyl alcohol, linaloyl oxide, p-linalool, allo-ocimenol, and 3,7-dimethyl-1,6-octadien-3-ol.Linalool has a stereogenic center at C3 and therefore there are two stereoisomers: (R)-(−)-linalool is also known as licareol and (S)-(+)-linalool is also known as coriandrol. Copolymers of the two stereoisomers produce unique compounds useful in cosmetic applications.

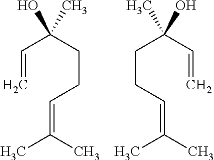

(S)-(+)-linalool (left) and (R)-(−)-linalool (right)

Although both enantiomeric forms are found in nature they are never found copolymerized. (S)-linalool is found, for example, as a major constituent of the essential oils of coriander (Coriandrum sativum L. family Apiaceae) seed, palmarosa [Cymbopogon martinii var martinii (Roxb.) Wats., family Poaceae], and sweet orange (Citrus sinensis Osbeck, family Rutaceae) flowers. (R)-linalool is present in lavender (Lavandula officinalis Chaix, family Lamiaceae), bay laurel (Laurus nobilis, family Lauraceae), and sweet basil (Ocimum basilicum, family Lamiaceae), among others.

Each enantiomer evokes different neural responses in humans, and therefore are classified as possessing distinct scents. (S)-(+)-Linalool is perceived as sweet, floral, petitgrain-like (odor threshold 7.4 ppb) and the (R)-form as more woody and lavender-like (odor threshold 0.8 ppb).

Phenethyl alcohol, or 2-phenylethanol, is an organic compound that consists of a phenethyl group (C₆H₅CH₂CH₂) group attached to OH. The OH group makes it amenable to copolymerization. It is a colourless liquid that is slightly soluble in water (2 ml/100 ml H2O), but miscible with most organic solvents. It occurs widely in nature, and is found in a variety of essential oils. It has a pleasant floral odor.

Farnesol is a natural 15-carbon organic compound which is an acyclic sesquiterpene alcohol. Under standard conditions, it is a colorless liquid. It is hydrophobic, and thus insoluble in water, but miscible with oils.

Farnesol is produced from 5-carbon isoprene compounds in both plants and animals. Phosphate activated derivatives of farnesol are the building blocks of most, and possibly all, acyclic sesquiterpenoids. These compounds are doubled to form 30-carbon squalene, which in turn is the precursors for steroids in plants, animals, and fungi. As such, farnesol and its derivatives are important starting compounds for both natural and artificial organic synthesis.

Farnesol is present in many essential oils such as citronella, neroli, cyclamen, lemon grass, tuberose, rose, musk, balsam and tolu. It is used in perfumery to emphasize the odors of sweet floral perfumes. Its method of action for enhancing perfume scent is as a co-solvent that regulates the volatility of the odorants. It is especially used in lilac perfumes.

Farnesol is a natural pesticide for mites and is a pheromone for several other insects. Copolymers of the present invention can enhance pesticidal activity by combining compounds that have complementary effect on pests. Copolymers can also provide a broader spectrum of activity.

Eugenol is a phenylpropene, an allyl chain-substituted guaiacol. Eugenol is a member of the phenylpropanoids class of chemical compounds. It is a colourless to pale yellow, aromatic oily liquid extracted from certain essential oils especially from clove oil, nutmeg, cinnamon, basil and bay leaf. It is present in concentrations of 80-90% in clove bud oil and at 82-88% in clove leaf oil.

Eugenol is used in perfumes, flavorings, and essential oils. It is also used as a local antiseptic and anaesthetic. Eugenol can be combined with zinc oxide to form zinc oxide eugenol which has restorative and prosthodontic applications in dentistry. For example, zinc oxide eugenol is used for root canal sealing. Copolymers of the present invention also are benefited by the association with zinc oxide.

Attempts have been made to develop eugenol derivatives as intravenous anesthetics, as an alternative to propanidid which produces unacceptable side effects around the site of injection in many patients. It can be used to reduce the presence of Listeria monocytogenes and Lactobacillus sakei in food.

Benzyl alcohol is an aromatic alcohol with the formula C₆H₅CH₂OH. Benzyl alcohol is a colorless liquid with a mild pleasant aromatic odor. It is a useful solvent due to its polarity, low toxicity, and low vapor pressure. Benzyl alcohol has moderate solubility in water (4 g/100 mL) and miscible in alcohols and diethyl ether.

Benzyl alcohol is produced naturally by many plants and is commonly found in fruits and teas. It is also found in a variety of essential oils including jasmine, hyacinth, and ylang-ylang.

Pomegranate Seed Oil: Punicic acid (also called trichosanic acid) is a polyunsaturated fatty acid, 18:3 cis-9, trans-11, cis-13. It is named for the pomegranate, (Punica granatum), and is obtained from pomegranate seed oil. Punicic acid is a conjugated linolenic acid, i.e. it has three conjugated double bonds. It is chemically similar to the conjugated linoleic acids which have two. OLETF rats—a strain which becomes obese—remained relatively lean when punicic acid was added to their feed.

Peppermint oil: Peppermint oil has a high concentration of natural pesticides, mainly pulegone (found mainly in Mentha arvensis var. piperascens Cornmint, Field Mint, Japanese Mint and to a lesser extent in Mentha×piperita subsp. nothosubsp. piperita) and menthone.

The chemical constituents of the essential oil from peppermint (Mentha x piperita L.) include menthol (40.7%) and menthone (23.4%). Other components are (+/−)-menthyl acetate, 1,8-cineole, limonene, beta-pinene and beta-caryophyllene.

Pulegone is a naturally occurring organic compound obtained from the essential oils of a variety of plants such as Nepeta cataria (catnip), Mentha piperita, and pennyroyal. It is classified as a monoterpene. Pulegone is a clear colorless oily liquid and has a pleasant odor similar to pennyroyal, peppermint and camphor. It is used in flavoring agents, in perfumery, and in aromatherapy.

Menthol is an organic compound made synthetically or obtained from corn mint, peppermint, or other mint oils. It is a waxy, crystalline substance, clear or white in color, which is solid at room temperature and melts slightly above. The main form of menthol occurring in nature is (−)-menthol. Menthol has local anesthetic and counterirritant qualities, and it is widely used to relieve minor throat irritation. Menthol also acts as a weak kappa opioid receptor agonist. Other Oils

Other naturally found oils useful in the synthesis of the copolymers of the present invention include: almond oil, borage oil, canola oil, castor oil, coconut oil, corn oil, cottonseed oil, grape seed oil, hemp oil, mango oil, jojoba oil, mustard oil, neem oil, palm kernel oil, rapeseed oil, safflower oil, sesame oil, shea butter, sunflower oil, and tonka bean oil.

General categories of oils useful in the synthesis of the copolymers of oils of the present invention include: omega-3 fatty acids such as hexadecatrienoic acid (all-cis 7,10,13-hexadecatrienoic acid), stearidonic acid (all-cis-6,9,12,15,-octadecatetraenoic acid), eicosatrienoic acid (all-cis-11,14,17-eicosatrienoic acid), eicosatetraenoic acid (all-cis-8,11,14,17-eicosatetraenoic acid), eicosapentaenoic acid (all-cis-5, 8,11,14,17-eicosapentaenoic acid), heneicosapentaenoic acid (all-cis-6,9,12,15,18-heneicosapentaenoic acid), docosapentaenoic acid (all-cis-7,10,13,16,19-docosapentaenoic acid), docosahexaenoic acid (all-cis-4,7,10,13,16,19-docosahexaenoic acid), tetracosapentaenoic acid (all-cis-9,12,15,18,21-tetracosapentaenoic acid), tetracosahexaenoic acid (all-cis-6,9,12,15,18,21-tetracosahexaenoic acid); and omega-6 fatty acids including gamma-linolenic acid (all-cis-6,9,12-octadecatrienoic acid), eicosadienoic acid (all-cis-11,14-eicosadienoic acid), dihomo-gamma-linolenic acid (all-cis-8,11,14-eicosatrienoic acid), arachidonic acid (all-cis-5, 8,11,14-eicosatetraenoic acid), docosadienoic acid (all-cis-13,16-docosadienoic acid), adrenic acid (all-cis-7,10,13,16-docosatetraenoic acid), docosapentaenoic acid (all-cis-4,7,10,13,16-docosapentaenoic acid), tetracosatetraenoic acid (all-cis-9,12,15,18-tetracosatetraenoic acid), tetracosapentaenoic acid (all-cis-6,9,12,15,18-tetracosapentaenoic acid); and omega-9 fatty acids including oleic acid (cis-9-octadecenoic acid), eicosenoic acid (cis-11-eicosenoic acid), mead acid (all-cis-5,8,11-eicosatrienoic acid), erucic acid (cis-13-docosenoic acid), nervonic acid (cis-15-tetracosenoic acid); and conjugated fatty acids such as rumenic acid (9Z,11E-octadeca-9,11-dienoic acid), α-Calendic acid (8E,10E,12Z-octadecatrienoic acid), β-calendic acid (8E,10E,12E-octadecatrienoic acid), jacaric acid (8Z,10E,12Z-octadecatrienoic acid), α-eleostearic acid (9Z,11E,13E-octadeca-9,11,13 -trienoic acid), β-eleostearic acid (9E,11E,13E-octadeca-9, 11,13 -trienoic acid), catalpic acid (9Z,11Z,13E-octadeca-9,11,13-trienoic acid), punicic acid (9Z, 11E,13Z-octadeca-9, 11,13-trienoic acid). Other useful oils are: rumelenic acid (9E,11Z,15E-octadeca-9,11,15-trienoic acid), α-parinaric acid (9E,11Z,13Z,15E-octadeca-9,11,13,15-tetraenoic acid), β-parinaric acid (trans-octadeca-9,11,13,15-tretraenoic acid), bosseopentaenoic acid (5Z,8Z,10E,12E,14Z-eicosanoic acid), pinolenic acid ((5Z,9Z,12Z)-octadeca-5,9,12-trienoic acid), Podocarpic acid ((5Z,11Z,14Z)-eicosa-5,11,14-trienoic acid), Linker Molecules

For the purposes of the invention, the term “linker molecule” means any molecule with pendant functional groups comprising groups that are H bond donors or acceptors, and that are capable of establishing at least 3 urethane/urea type bonds with oil molecules or proteins. The linker may comprise the structure —R¹—O—C(O)—NH—R²—NCO, as defined above.

Preferably, the di-oil synthesis is carried out in the absence of water so that chain extension does not occur between linker molecules, and the covalent reactions are exclusively with the OH or NH₂ groups of oil molecules. Preferably, on each linker molecule is bonded two oil molecules to two of the functional groups of the linker molecule, and the remaining unreacted functional group of the linker molecule remains unreacted in the finished composition.

The radicals R¹ and R² are independently may especially be: a linear or branched, divalent C2-C12 alkylene group, especially a 1,2-ethylene, 1,6-hexylene, 1,4-butylene, 1,6-(2,4,4-trimethylhexylene), 1,4-(4-methylpentylene), 1,5-(5-methylhexylene), 1,6-(6-methylheptylene), 1,5-(2,2,5-trimethylhexylene) or 1,7-(3,7-dimethyloctylene) group; a divalent C4-C12 cycloalkylene or arylene group, chosen especially from the following radicals: isophorone, tolylene, 2-methyl-1,3-phenylene, 4-methyl-1,3-phenylene; 4,4′-methylenebiscyclohexylene; and 4,4-bisphenylenemethylene.

Preferably R¹ is a poloxamer. Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade names Multranol, Synperonics, Pluronics, and Kolliphor.

The lengths of the polymer blocks can be customized, and their association with human tissue can be customized accordingly. The hydrophobic blocks aid in the association of the di-oil molecule with keratin, whereas the hydrophilic aspect can associate with water, providing repaired keratin structure with an affinity for hydration.

Many different poloxamers exist that have slightly different properties. For the generic term “poloxamer”, these copolymers are commonly named with the letter “P” (for poloxamer) followed by three digits: the first two digits ×100 give the approximate molecular mass of the polyoxypropylene core, and the last digit ×10 gives the percentage polyoxyethylene content (e.g. P407=poloxamer with a polyoxypropylene molecular mass of 4,000 g/mol and a 70% polyoxyethylene content). Poloxamers with polyoxyethylene content of between 50% and 80% are preferred. Most preferred are poloxamers with 75% polyoxyethylene content and 25% polyoxypropylene content. Poloxamers with 3 to 6 arms are preferred, more preferably 3 to 4 arms.

An important characteristic of poloxamer solutions, and di-oils synthesized from them (poloxamer di-oils), is their temperature dependent self-assembling and thermo-gelling behavior. When poloxamer di-oils are applied to wet hair, a concentrated aqueous solutions of poloxamer di-oil is liquid at low temperature and form a gel at higher temperature in a reversible process. The liquid phase aids in delivery to hair, and the gelling process provides a more luxurious feel when used as an in-shower treatment.

The transition points that occur in these systems depend on the polymer composition (molecular weight and hydrophilic/hydrophobic molar ratio). At low temperatures and concentrations (below the critical micelle temperature and critical micelle concentration) individual block copolymers are present in solution. Above these values, aggregation of individual unimers occurs in a process called micellization forming supramolecules.

This aggregation is driven by the dehydration of the hydrophobic polyoxypropylene block that becomes progressively less soluble as the polymer concentration or temperature increases. The aggregation of several di-oil molecules occurs to minimize the interactions of the PPO blocks with the water. Thus, the core of the aggregates is made from the insoluble blocks (polyoxypropylene) while the soluble portion (polyoxyethylene) forms the shell of the micelles.

Compositions of di-oils of the present invention may be enhanced, or customized to particular tissue types, by employing poloxamers not functionalized by isocyanate or imidazole as a carrier for the di-oils.

The mechanisms on the micellization at equilibrium have shown to depend on two relaxation times: (1) the first and fastest (tens of the microseconds scale) corresponds to poloxamer di-oil exchange between micelles and the bulk solution (possibly containing free poloxamers) and follows the Aniansson-Wall model, and (2) the second and much slower one (in the millisecond range) is attributed to the formation and breakdown of whole micellar units leading to the final micellar size equilibration.

Although these mechanism are fast compared to a typical treatment time, their separation in time governs how protein bonds form and in what geometry. For example, the geometry of the cuticle can interact advantageously with the geometry of the micellar structure to promote infiltration and closure of the keratin structure of the hair cuticle.

Besides spherical micelles, elongated or wormlike micelles can also be formed. The final geometry will depend on the entropy costs of stretching the blocks, which is directly related to their composition (size and polyoxypropylene/polyoxyethylene ratio). For example, micelles can be formed that wrap around a hair shaft preferentially rather than remain in the spaces between hair shafts. This property can allow fewer di-oil molecules per unit volume of product, decreasing the cost of the composition, and enhancing the product feel.

The mechanisms involved in the shape transformation are different compared to the dynamics of micellization. For example, the mechanisms that causes a sphere-to-rod transition of block copolymer micelles, in which the micellar growth can occur by fusion/fragmentation of micelles or concomitant fusion/fragmentation of micelles and poloxamer di-oil exchange, is followed by smoothing of the rod-like structures. A protein bonding di-oil arranged in a rod geometry, will align along the direction of the keratin structures providing more effective coverage and repair by sealing the interstitial space between keratin structures.

With higher increments of the temperature and/or concentration, other phenomenon can occur such as the formation of highly ordered mesophases (cubic, hexagonal and lamellar). Eventually, a complete dehydration of the polyoxypropylene blocks and the collapse of the polyoxyethylene chains will lead to clouding and/or macroscopic phase separation. This is due to the fact that hydrogen bonding between the polyoxyethylene and the water molecules breaks down at high temperature and polyoxyethylene becomes also insoluble in water.

These self-assembly features provide the supramolecular structure at point of application, even when no such structure occurs in the product prior to use. This point of application self-assembly occurs especially when, typically, the product is non aqueous, and the application environment contains water.

The phase transitions can also be largely influenced by the use of additives such as salts and alcohols, commonly found in beauty products. The interactions with salts are related to their ability to act as water structure makers or water structure breakers. Water structure makers increase the self-hydration of water through hydrogen bonding and reduce the hydration of the poloxamer di-oil copolymers, thus reducing the critical micelle temperature and critical micelle concentration. Water structure breakers are electrolytes that reduce the water self-hydration and increase the polymer hydration, therefore increasing the critical micelle temperature and critical micelle concentration.

The linker molecules of the present invention preferably possess an amphiphilic structure. The linker molecules and di-oil constructed from them have surfactant properties that make them useful in hair treatment applications. Among other things, they can be used to increase the water solubility of hydrophobic, oily substances or otherwise increase the miscibility of two substances with different hydrophobicities. This feature is especially advantageous when amphiphilic poloxamers are joined with oils. The wettability of these oils is greatly enhanced by covalently bonding them to an amphiphilic core.

When mixed with water, concentrated solutions of poloxamers can form hydrogels. These gels can be extruded easily, acting as a carrier for other less soluble constituents. Di-oils of the present invention can be made into gels. Gelled di-oils have a substantive feel while not feeling soapy, and can be cleared from the hair after the treatment period without leaving a soapy residue.

Poloxamers and polyether diols useful in the present invention include: 1) Multranol 3901, a polyether polyol with functionality 3, typical OH number 28 mg KOH/g, typical molecular weight 6,000; typical viscosity 1,160 mPa-·s @ 25° C.; 2) Multranol 4011, functionality of 3; typical OH number 550 mg KOH/g; typical molecular weight 306; typical viscosity 1,650 mPa-·s @ 25° C.; and 3) Multranol 4012, functionality of 3; typical OH no. 370mg KOH/g; typical molecular weight 455; typical viscosity 650 mPa-·s @ 25° C.

Poloxamer triols can be synthesized by reacting trimethylol propane, or any small molecular weight triol, with poloxamer diols and diisocyanate, where the triol and the diols are covalently bonded via urethane links formed between the OH of the diol and triol and the isocyanate groups of the diisocyanate.

Acidic/basic conditions can also affect supramolecular structure. Especially when the di-oils are mixed with pH sensitive polymers. pH responsive dendrimers include poly-amidoamide dendrimers, poly(propyleneimine) dendrimers, Poly(L-lysine) ester, Poly(hydroxyproline), Poly(propyl acrylic acid), Poly(methacrylic acid), Polysilamine, Poly(methacrylic acid), PMAA-PEG copolymer, Maleic anhydride, and N,N-dimethylaminoethyl methacrylate.

Di-oils can be mixed with temperature sensitive polymers. Temperature sensitive polymers include poloxamers, prolastin, poly(N-substituted acrylamide), poly(organophosphazene), cyclotriphosphazenes with poly(ethyleneglycol) and amino acid esters, block copolymers of poly(ethylene glycol)/poly(lactic-co-glycolic acid), Poly(ethylene glycol), Poly(propylene glycol), PMAA, Poly(vinyl alcohol), various silk-elastin-like polymers, Poly(silamine), Poly(vinyl methyl ether), Poly(vinyl methyl oxazolidone), Poly(vinyl pyrrolidone), Poly(N-vinylcaprolactam), poly(N-vinyl isobutyl amid), poly(vinyl methyl ether), poly(N-vinylcaprolactam), Poly(siloxyethylene glycol), poly(dimethylamino ethyl methacrylate), triblock copolymer poly(DL-lactide-co-glycolide-b-ethylene glycol-b-DL-lactide-co-glycolide) (PLGA-PEG-PLGA), Cellulose derivatives, Alginate, Gellan, and Xyloglucan.

The term “copolymer” denotes polymers of oil molecules derived from at least two oil molecules of different chemical structure. The term “copolymers” thus also includes terpolymers, tetrapolymers and larger polymers of oil molecule units.

The di-oils of the present invention have at least one of the following structures: Urea Linkage

L-N═C═O+M-NH₂→L (NH)-(C═O)-(NH)-M

Urethane linkage

L-N═C═O+M-OH→L-(NH)-(C═O)—O-M

Carbamate linkage

L-O—(C═O)—N—(C═N)₂+M-NH₂→L-O—(C═O)-(NH)-M

Amide linkage

L-N═C═O+M-(C═O)OH →L-(NH)-(C═O)-M

where L is a linker backbone and M is an oil. The oil M has either an —OH, —(C═O)OH or NH₂ group as shown.

Typical di-oils in some embodiments may have of the have one of the following structures:

M(NH)-(C═O)-(NH)L(N═C═O)(NH)-(C═O)-(NH)-M

MO-(C═O)-(NH)L(N═C═O)(NH)-(C═O)-O-M

M(NH)-(C═O)-OL(N═C═O)-O-(C═O)-(NH)-M

M(C═O)-(NH)L(N═C═O)(NH)-(C═O)-M

Combinations of amide, urea, urethane and carbamate linkages are possible. For example, by using one oil having an OH group and another oil having a carboxylic acid group, the following structure can be formed:

MO(C═O)-(NH)L(N═C═O)(NH)-(C═O)-M

Di-oils with any ratio L/M>=2, L and M integers are possible. M is any oil molecule minus one OH or NH2 group. L is any n-ol, n>2, with the n OH groups replaced by n urethane links and n diisocyanate molecule minus two NCO groups. For example, a triol

(HO[(C₃H₆)—O]n)₃-(CH₂)—C-Et

is reacted with three toluene diisocyanates

O═C═N—(C₆H₄)—N═C═O

to yield a prepolymer having the structure:

L-(N═C═O)³

### EXAMPLES

### Example 1

### Di-Oil

A triisocyanate polyether polyol was synthesized by reacting one mole of PE/PO 80:20 random copolymer diol having an average MW of 2600 with two moles of BASF Luparnate T80-1 (80:20 2,4- and 2,6-toluene diisocyanate) and ¹/₄ mole of trimethylolpropane to obtain a NCO-terminated hydrophilic prepolymer having a free NCO content of 3%.

One third mole of the triisocyanate polyether polyol of above, was reacted with 1/9 mole of oleic acid (prickly pear oil) and 1/9 mole of 13-Hydroxyoctadecadienoic acid (argan oil) to yield a protein bonding di-oil.

### Example 2

### Di-Oil

A tri-functional polyether polyol was formed by reacting one mole of a PE/PO 80:20 random copolymer having an average MW of 2600 with two moles of isophorone diisocyanate (IPDI) and ⅓ mole trimethylolpropane to obtain a NCO-terminated hydrophilic prepolymer having a free NCO content of 1.5%.

One third mole of the triisocyanate polyether polyol of above, was reacted with 1/9 mole of Citronellol (rose oil) and 1/9 mole of trichosanic acid (pomegranate seed oil) to yield a protein bonding di-oil.

### Example 3

A tri-functional polyether polyol was formed by reacting one mole of a PE/PO 80:20 random copolymer having an average MW of 2600 and one mole of polyethylene glycol of 1000 MW with 4 moles of IPDI and ⅔ mole of trimethylolpropane (1-5%) to obtain a NCO-terminated hydrophilic prepolymer having a free NCO content of 1.5%.

One third mole of the triisocyanate polyether polyol of above, was reacted with 1/9 mole of linoleic acid (flax seed oil) and 1/9 mole of menthol (mint oil) to yield a protein bonding di-oil.

### Example 4

### Di-Oil

A tri-functional polyether polyol was formed by reacting one mole of a PE/PO 80:20 random copolymer having an average MW of 2600 and one mole of polyethylene glycol of 1000 MW with 4 moles of BASF Luparnate T80-1 (80:20 2,4- and 2,6-toluene diisocyanate) and ⅔ moles of trimethylolpropane (1-5%) to obtain a NCO-terminated hydrophilic prepolymer having a free NCO content of 1.5%.

One third mole of the triisocyanate polyether polyol of above, was reacted with 2/9 mole of linoleic acid (flax seed oil) and 1/9 mole of menthol (mint oil) to yield a non-protein bonding di-oil.

### Example 5

### Gel Di-Oil

In this example a polyether hydroxyl-terminated copolymer of 75% ethylene oxide and 25% propylene oxide is used as the triol. One equivalent of Multranol 9199 (3066 g) is combined with 3 equivalent of toluene diisocyanate (261 g) at room temperature (22 oC). The mixture is stirred at 100 revolutions per minute and the temperature monitored. The mixture will begin to heat up by exothermic reaction and no heat is to be applied to the reactor until the temperature in the reactor ceases to rise. Then the mixture temperature should be increased in 5 oC increments per ½ hour until the mixture reaches 60 oC. The reaction should be continued until the %NCO=1.3%. The target % NCO is reached when every hydroxyl group in the mixture is reacted with an NCO group. Ideally, the result is comprised of single triols endcapped with three diisocyanates. This outcome can be enhanced by slow addition of the triol to the diisocyanate. The addition should be in 10 g increments, added when the exotherm from the previous addition has ceased. However, chain extended variations of the above ideal outcome are useful, their primary disadvantage being that the product is slightly higher in viscosity. The ideal %NCO is calculated by dividing the weight of the functional isocyanate groups (3×42 Dalton) per product molecule by the total weight of the product molecule (9199 Dalton+3×174 Dalton) yielding approximately 1.3%.

To this triisocyanate poloxamer is added ½ equivalent linoleic acid and ¼ equivalent oleic acid and reacted at 70 oC until no OH functionality remains. Multranol 9199 is available from Bayer (Pittsburg, Pa.).

Thus, although there have been described particular embodiments of the present invention of a new and useful Tissue Bonding Co-Polymers of Oil it is not intended that such references be construed as limitations upon the scope of this invention except as set forth in the following claims.

What is claimed is:

1. A keratin care composition comprising, in a keratin care acceptable medium, a compound obtained by the reaction of: i) at least two oil molecules each containing at least one functional group selected from the group consisting of —OH, —C(O)OH, —NH₂, and combinations thereof and ii) at least one linking molecule comprising at least two isocyanate or imidazole groups capable of reacting with the reactive functional groups on the oil molecules, wherein the linking molecule further comprises at least one pendant group capable of bonding to a protein.

2. The composition of claim 1, wherein the linking molecule comprises three isocyanate groups.

3. The composition of claim 1, wherein the functional groups on the at least two oils are —OH, CO₂H or a combination thereof.

4. The composition of claim 1, wherein the at least two oils are the same or different and are selected from the group consisting of: a saturated or unsaturated, linear or branched C₆-C₅₀ monoalcohol; a saturated or unsaturated, linear or branched C₆C-₅₀ diol; a saturated or unsaturated, linear or branched C₆-C₅₀ triol; linear or branched C₆-C₅₀ fatty acid; a pentaerythritol partial ester; a dipentaerythritol diester, triester, tetraester, or pentaester.

5. The composition of claim 1, wherein the linking molecule comprises a backbone molecule selected from the group consisting of: trimethylolpropane monoester, trimethylolpropane monoester diester; a bis(trimethylolpropane) monoester, a bis(trimethylolpropane) monoester diester, a bis(trimethylolpropane) monoester triester; an ester of glycerol, an ester of polyglycerol; a propylene glycol monoester; a diol dimer monoester; a glycerol ether; an ester of a hydroxylated monocarboxylic acid, an ester of a hydroxylated monocarboxylic an ester of a dicarboxylic acid, an ester of a hydroxylated tricarboxylic acid; a triglyceryl ester comprising an OH; a hydrogenated castor oil, a non-hydrogenated castor oil, and a poloxamer.

6. The composition of claim 5, wherein the linking molecule comprises three terminal groups, two terminal groups of which are bonded to the oil molecules, and the third terminal group is a free isocyanate group.

7. The composition of claim 1, wherein the at least one protein bonding terminal group is an isocyanate group derived by the reaction of an —OH or —NH₂ group on the linker molecule with toluene diisocyanate, isophorone diisocyanate, methylenebis(phenyl isocyanate), hexamethylene diisocyanate, naphthalene diisocyanate, and methylene bis-cyclohexylisocyanate.

8. The composition of claim 1, wherein the oil has a refractive index of greater than or equal to 1.46 at 25° C.

9. The composition of claim 1, wherein the oil has a molecular weight (Mw) ranging from 150 to 6000 g/mol.

10. The composition of claim 1, wherein the oil is selected from the group consisting of: a linear, branched, or cyclic, saturated or unsaturated fatty alcohol comprising 6 to 50 carbon atoms and an OH group, and optionally an NH₂; and an ester of a hydroxylated dicarboxylic acid with a monoalcohol.

11. The composition of claim 1, wherein the oils are the same or different and are selected from the group consisting of argan oil, oleic acid, citronellol, trichosanic acid, linoleic acid and menthol.

12. The composition of claim 1, wherein, the linker molecule contains the radical —R¹—O—C(O)—NH—R²—NCO, wherein R¹ and R² are each independently a divalent carbon-based radical selected from the group consisting of a linear or branched alkyl group, a cycloalkyl group, and an aryl group; or a mixture thereof.

13. The composition of claim 1, wherein the linker is obtained by the reaction of a polyethylene/polypropylene copolymer with at least three diisocyanate molecules.

14. The composition of claim 13, wherein the copolymer is a poloxamer.

15. The composition of claim 1, wherein the number-average molecular mass (Mn) of the compound is from 180 to 8000.

16. The composition of claim 1, wherein the compound is present in the composition in an amount ranging from 5% to 95% by weight, relative to a total weight of the composition.

17. The composition of claim 1, wherein the composition further comprises a semicrystalline polymer selected from the group consisting of: a homopolymer or copolymer comprising a unit obtained from polymerizing a monomer comprising a crystallizable hydrophobic side chain; a polymer comprising, in the backbone, a crystallizable block; a polycondensate of an aliphatic, an aromatic, or an aliphatic/aromatic polyester; at least one selected from the group consisting of an ethylene homopolymer, a propylene homopolymer, and an ethylene/propylene copolymer prepared via metallocene catalysis.

18. The composition of claim 1, wherein the composition further comprises a thickener selected from the group consisting of: (i) a polymer having a weight-average molecular mass of less than 100,000, and comprising a) a polymer backbone comprising hydrocarbon repeating units comprising a heteroatom, and optionally comprising b) at least one selected from the group consisting of a pendent fatty chain and a terminal fatty chain, optionally functionalized, comprising from 6 to 120 carbon atoms, and linked to the hydrocarbon; and (ii) a silicone polyamide.

19. The composition of claim 1, further comprising: at least one additive selected from the group consisting of a volatile oil, a nonvolatile oil, a dyestuff, a pasty fatty substance, a wax, and a filler.

20. A method for treating a keratin material, the process comprising: applying the cosmetic composition of claim 1 to a keratin material.

21. A keratin care composition comprising, in a keratin care acceptable medium, a compound obtained by the reaction of: i) at least two oil molecules each containing at least one functional group selected from the group consisting of —OH, —C(O)OH, —NH₂, and combinations thereof and ii) at least one linking molecule comprising at least two isocyanate or imidazole groups capable of reacting with the reactive functional groups on the oil molecules.

22. The composition of claim 21, wherein the linking molecule comprises three isocyanate groups, each of which are bonded to the oil molecules.

23. The composition of claim 21, wherein the functional groups on the at least two oils are —OH, —CO₂H or a combination thereof.

24. The composition of claim 21, wherein the at least two oils are the same or different and are selected from the group consisting of: a saturated or unsaturated, linear or branched C₆C-₅₀ monoalcohol; a saturated or unsaturated, linear or branched C₆C-₅₀ diol; a saturated or unsaturated, linear or branched C₆-C₅₀ triol; linear or branched C₆-C₅₀ fatty acid; a pentaerythritol partial ester; a dipentaerythritol diester, triester, tetraester, or pentaester.

25. The composition of claim 21, wherein the linking molecule comprises a backbone molecule selected from the group consisting of: trimethylolpropane monoester, trimethylolpropane monoester diester; a bis(trimethylolpropane) monoester, a bis(trimethylolpropane) monoester diester, a bis(trimethylolpropane) monoester triester; an ester of glycerol, an ester of polyglycerol; a propylene glycol monoester; a diol dimer monoester; a glycerol ether; an ester of a hydroxylated monocarboxylic acid, an ester of a hydroxylated monocarboxylic an ester of a dicarboxylic acid, an ester of a hydroxylated tricarboxylic acid; a triglyceryl ester comprising an OH; a hydrogenated castor oil, a non-hydrogenated castor oil, and a poloxamer.

26. The composition of claim 21, wherein the oil has a refractive index of greater than or equal to 1.46 at 25° C.

27. The composition of claim 21, wherein the oil has a molecular weight (Mw) ranging from 150 to 6000 g/mol.

28. The composition of claim 21, wherein the oil is selected from the group consisting of: a linear, branched, or cyclic, saturated or unsaturated fatty alcohol comprising 6 to 50 carbon atoms and an OH group, and optionally an NH₂; and an ester of a hydroxylated dicarboxylic acid with a monoalcohol.

29. The composition of claim 21, wherein the oils are the same or different and are selected from the group consisting of argan oil, oleic acid, citronellol, trichosanic acid, linoleic acid and menthol.

30. The composition of claim 21, wherein, the linker molecule contains the radical —R¹—O—C(O)—NH—R²—NCO, wherein R¹ and R² are each independently a divalent carbon-based radical selected from the group consisting of a linear or branched alkyl group, a cycloalkyl group, and an aryl group; or a mixture thereof.

31. The composition of claim 21, wherein the linker is obtained by the reaction of a polyethylene/polypropylene copolymer with at least three diisocyanate molecules.

32. The composition of claim 31, wherein the copolymer is a poloxamer.

33. The composition of claim 21, wherein the compound does not contain free isocyanate or imidazole groups.

34. The composition of claim 21, wherein the number-average molecular mass (Mn) of the compound is from 180 to 8000.

35. The composition of claim 21, wherein the compound is present in the composition in an amount ranging from 5% to 95% by weight, relative to a total weight of the composition.

36. The composition of claim 21, wherein the composition further comprises a semicrystalline polymer selected from the group consisting of: a homopolymer or copolymer comprising a unit obtained from polymerizing a monomer comprising a crystallizable hydrophobic side chain; a polymer comprising, in the backbone, a crystallizable block; a polycondensate of an aliphatic, an aromatic, or an aliphatic/aromatic polyester; at least one selected from the group consisting of an ethylene homopolymer, a propylene homopolymer, and an ethylene/propylene copolymer prepared via metallocene catalysis.

37. The composition of claim 21, wherein the composition further comprises a thickener selected from the group consisting of: (i) a polymer having a weight-average molecular mass of less than 100,000, and comprising a) a polymer backbone comprising hydrocarbon repeating units comprising a heteroatom, and optionally comprising b) at least one selected from the group consisting of a pendent fatty chain and a terminal fatty chain, optionally functionalized, comprising from 6 to 120 carbon atoms, and linked to the hydrocarbon; and (ii) a silicone polyamide.

38. The composition of claim 21, further comprising: at least one additive selected from the group consisting of a volatile oil, a nonvolatile oil, a dyestuff, a pasty fatty substance, a wax, and a filler.

39. A method for treating a keratin material, the process comprising: applying the cosmetic composition of claim 21 to a keratin material.

40. A keratin care composition comprising, in a keratin care acceptable medium, a supramolecular compound obtained by the reaction of: i) at least two oil molecules each containing at least one functional group selected from the group consisting of —OH, —C(O)OH, —NH₂, and combinations thereof and ii) at least one linking molecule comprising at least two isocyanate or imidazole groups capable of reacting with the reactive functional groups on the oil molecules, wherein the linking molecule further comprises at least one pendant group capable of bonding to a protein.

41. The composition of claim 40, wherein the linking molecule comprises three isocyanate groups.

42. The composition of claim 40, wherein the functional groups on the at least two oils are —OH, CO₂H or a combination thereof.

43. The composition of claim 40, wherein the at least two oils are the same or different and are selected from the group consisting of: a saturated or unsaturated, linear or branched C₆C-₅₀ monoalcohol; a saturated or unsaturated, linear or branched C₆C-₅₀ diol; a saturated or unsaturated, linear or branched C₆-C₅₀ triol; linear or branched C₆-C₅₀ fatty acid; a pentaerythritol partial ester; a dipentaerythritol diester, triester, tetraester, or pentaester.

43. The composition of claim 40, wherein the linking molecule comprises a backbone molecule selected from the group consisting of: trimethylolpropane monoester, trimethylolpropane monoester diester; a bis(trimethylolpropane) monoester, a bis(trimethylolpropane) monoester diester, a bis(trimethylolpropane) monoester triester; an ester of glycerol, an ester of polyglycerol; a propylene glycol monoester; a diol dimer monoester; a glycerol ether; an ester of a hydroxylated monocarboxylic acid, an ester of a hydroxylated monocarboxylic an ester of a dicarboxylic acid, an ester of a hydroxylated tricarboxylic acid; a triglyceryl ester comprising an OH; a hydrogenated castor oil, a non-hydrogenated castor oil, and a poloxamer.

44. The composition of claim 43, wherein the linking molecule comprises three terminal groups, two terminal groups of which are bonded to the oil molecules, and the third terminal group is a free isocyanate group.

45. The composition of claim 40, wherein the at least one protein bonding terminal group is an isocyanate group derived by the reaction of an —OH or —NH₂ group on the linker molecule with toluene diisocyanate, isophorone diisocyanate, methylenebis(phenyl isocyanate), hexamethylene diisocyanate, naphthalene diisocyanate, and methylene bis-cyclohexylisocyanate.

46. The composition of claim 40, wherein the oil has a refractive index of greater than or equal to 1.46 at 25° C.

47. The composition of claim 40, wherein the oil has a molecular weight (Mw) ranging from 150 to 6000 g/mol.

48. The composition of claim 40, wherein the oil is selected from the group consisting of: a linear, branched, or cyclic, saturated or unsaturated fatty alcohol comprising 6 to 50 carbon atoms and an OH group, and optionally an NH₂; and an ester of a hydroxylated dicarboxylic acid with a monoalcohol.

49. The composition of claim 40, wherein the oils are the same or different and are selected from the group consisting of argan oil, oleic acid, citronellol, trichosanic acid, linoleic acid and menthol.

50. The composition of claim 40, wherein, the linker molecule contains the radical —R¹—O—C(O)—NH—R²—NCO, wherein R¹ and R² are each independently a divalent carbon-based radical selected from the group consisting of a linear or branched alkyl group, a cycloalkyl group, and an aryl group; or a mixture thereof.

51. The composition of claim 40, wherein the linker is obtained by the reaction of a polyethylene/polypropylene copolymer with at least three diisocyanate molecules.

52. The composition of claim 51, wherein the copolymer is a poloxamer.

53. The composition of claim 40, wherein the number-average molecular mass (Mn) of the compound is from 180 to 8000.

54. The composition of claim 40, wherein the compound is present in the composition in an amount ranging from 5% to 95% by weight, relative to a total weight of the composition.

55. The composition of claim 40, wherein the composition further comprises a semicrystalline polymer selected from the group consisting of: a homopolymer or copolymer comprising a unit obtained from polymerizing a monomer comprising a crystallizable hydrophobic side chain; a polymer comprising, in the backbone, a crystallizable block; a polycondensate of an aliphatic, an aromatic, or an aliphatic/aromatic polyester; at least one selected from the group consisting of an ethylene homopolymer, a propylene homopolymer, and an ethylene/propylene copolymer prepared via metallocene catalysis.

56. The composition of claim 40, wherein the composition further comprises a thickener selected from the group consisting of: (i) a polymer having a weight-average molecular mass of less than 100,000, and comprising a) a polymer backbone comprising hydrocarbon repeating units comprising a heteroatom, and optionally comprising b) at least one selected from the group consisting of a pendent fatty chain and a terminal fatty chain, optionally functionalized, comprising from 6 to 120 carbon atoms, and linked to the hydrocarbon; and (ii) a silicone polyamide.

57. The composition of claim 40, further comprising: at least one additive selected from the group consisting of a volatile oil, a nonvolatile oil, a dyestuff, a pasty fatty substance, a wax, and a filler.

58. A method for treating a keratin material, the process comprising: applying the cosmetic composition of claim 40 to a keratin material.
